(19) United States
(12) Patent Application Publication
(10) Pub. No.: US 2010/0023112 A1
(43) Pub. Date: Jan. 28, 2010

(54) BIOCORRODIBLE IMPLANT WITH A COATING COMPRISING A HYDROGEL

(75) Inventors: Alexander Borck, Aurachtal (DE); Nina Adden, Nuernberg (DE)

Correspondence Address: BIOTECH BEACH LAW GROUP , PC, 5677 OBERLINE DRIVE, SUITE 204, SAN DIEGO, CA, 92121, US

(73) Assignee: BIOTRONIK VI PATENT AG, Baar (CH)

(21) Appl. No.: 12/507,216

(22) Filed: Jul. 22, 2009

(30) Foreign Application Priority Data
Jul. 28, 2008 (DE) 10 2008 040 787.9

(51) Int. Cl. A61F 2/06

(52) U.S. Cl. 623/1.15; 623/1.42

(57) ABSTRACT

The invention relates to an implant having a base body consisting completely or partially of a biocorrodible metallic material, the material being such that it decomposes in an aqueous environment to yield an alkaline product and the base body has a coating comprising or containing a hydrogel, characterized in that the hydrogel has a reduced swelling capacity at an elevated pH.

### CROSS-REFERENCE TO RELATED APPLICATIONS

This application claims benefit of priority to German patent application number DE 10 2008 040 787.9, filed Jul. 28, 2008; the contents of which are herein incorporated by reference in their entirety.

### TECHNICAL FIELD

The invention relates to a biocorrodible implant with a coating including a hydrogel.

### BACKGROUND OF THE INVENTION

Implants are used in a variety of embodiments in modern medical technology. For example, they are used to support blood vessels, hollow organs and duct systems (endovascular implants), for fastening and temporary fixation of tissue implants and tissue transplants but also for orthopedic purposes, e.g., as nails, plates or screws.

For example, implantation of stents has become established as one of the most effective therapeutic measures in treatment of vascular diseases. The purpose of stents is to assume a supporting function in a patient's hollow organs. Stents of a traditional design therefore have a filigree supporting structure comprising metallic struts, which are initially in a compressed form for introducing them into the body and then are expanded at the site of application. One of the main indications for use of such stents is for permanent or temporary dilatation and maintaining the patency of vasoconstrictions, in particular stenoses of coronary vessels. In addition, there are also known aneurysm stents, for example, which serve to support damaged vascular walls.

Stents have a circumferential wall of a sufficient supporting strength to keep the constricted vessel open to the desired extent, and have a tubular base body through which the blood continues to flow unhindered. The circumferential wall is usually formed by a mesh-like supporting structure, allowing the stent to be inserted in a compressed form with a small outside diameter as far the stenosis in the respective vessel to be treated, and to widen it there, e.g., with the help of a balloon catheter, to the extent that the vessel has the desired dilated inside diameter. A cardiologist must monitor the procedure of positioning and expansion of stents and the final position of the stent in the tissue after the end of the procedure. This can be accomplished by imaging methods, e.g., by radiology.

The implant or the stent has a base body of an implant material. An implant material is a nonliving material, which is used for an application in medicine and interacts with biological systems. The basic prerequisites for use of a material as an implant material that comes in contact with the physiological environment when used as intended is its biocompatibility. Biocompatibility is understood to be the ability of a material to induce an appropriate tissue reaction in a specific application. This includes adaptation of the chemical, physical, biological and morphological surface properties of an implant to the recipient tissue with the goal of achieving a clinically desired interaction. The biocompatibility of the implant material also depends on the chronological course of the reaction of the biosystem in the implant. Relatively short-term irritations and inflammations occur and lead to tissue changes. Biological systems react differently, depending on the properties of the implant material. According to the reaction of the biosystem, implant materials can be subdivided into bioactive, bioinert and degradable/absorbable materials.

A biological reaction to polymeric, ceramic or metallic implant materials depends on the concentration, duration of action and how administered. The presence of an implant material often leads to inflammation reactions triggered by mechanical irritants, chemicals and metabolites. The inflammation process is usually accompanied by migration of neutrophilic granulocytes and monocytes through the vascular walls, migration of lymphocyte effector cells, forming specific antibodies to the inflammation irritant, activation of the complement system and the release of complement factors, which act as mediators, and ultimately the activation of blood coagulation. An immunologic reaction is usually closely associated with an inflammation reaction and may lead to sensitization and allergization. Known metallic allergens comprise, for example, nickel, chromium and cobalt, which are also used as alloy components in many surgical implants. One important problem stent implantation of stents in blood vessels is in-stent restenosis due to excessive neointimal growth induced by highly proliferating smooth arterial muscle cells and a chronic inflammation reaction.

One promising approach to solving this problem lies in the use of biocorrodible metals and their alloys as the implant material because a permanent supporting function by the stent is not usually necessary. Initially damaged body tissues regenerate. In DE 197 31 021 A1, for example, it is proposed that medical implants should be made of a metallic material, the main component being iron, zinc or aluminum, and/or an element from the group of alkali metals or alkaline earth metals. Especially suitable alloys based on magnesium, iron and zinc are described as especially suitable. Secondary components of the alloys may include manganese, cobalt, nickel, chromium, copper, cadmium, lead, tin, thorium, zirconium, silver, gold, palladium, platinum, silicon, calcium, lithium, aluminum, zinc and iron. In addition, it is known from DE 102 53 634 A1 that a biocorrodible magnesium alloy with a >90% magnesium content, 3.7-5.5% yttrium, 1.5-4.4% rare earth metals and <1% remainder is suitable in particular for production of an endoprosthesis, e.g., in the form of a self-expanding or balloon-expandable stent. Use of biocorrodibic metallic materials in implants should lead to a definite reduction in rejection reactions or inflammation reactions. Such biocorrodible implants and stents often have a coating or cavity filling with a suitable polymer.

One problem when using these biocorrodible implants consisting completely or partially of a metallic material is that the degradation products, which are formed and eluted in corrosion of the implant, often have a significant influence on the local pH and thus can lead to unwanted tissue reactions as well as possibly having an adverse effect on the further corrosion rate of the implant. In degradation of biocorrodible implant materials containing Mg in particular, there may be an increase in the pH in the immediate vicinity. This increase in pH may lead to a phenomenon known as alkalosis. The local increase in pH results in an imbalance in charge distribution in muscle cells surrounding the blood vessel, which may lead to a local increase in muscle tone in the area of the implant. This increased pressure on the implant may lead to premature loss of implant integrity.

In addition, it is desirable to be able to influence the rate of corrosion of the biocorrodible implant and define it in advance in a targeted manner.

The object of the present invention was to reduce or overcome one or more of the disadvantages of the prior art described here.

### SUMMARY OF THE INVENTION

In one aspect of the present invention an implant with a base body is provided, which includes completely or partially a biocorrodible metallic material, where the material has properties such that it decomposes to form an alkaline product in an aqueous environment, and where the base body has a coating including or containing a hydrogel, characterized in that the hydrogel has a reduced swelling capacity at an elevated pH. In some embodiments, the implant is a stent. In some embodiments, the biocorrodible metallic material is a magnesium alloy.

In some embodiments, the swelling capacity of the hydrogel is reduced at a pH greater than 8. In related embodiments the swelling capacity of the hydrogel is reduced at a pH greater than 8, such that the hydrogel can absorb at least 30% less water than at a physiological pH. The hydrogel may have at least one functional group which is converted from an ionic charge state to a neutral charge state with an increase in pH. Further, exemplary functional groups include those that include an amine function or an amide function. The hydrogel may include a polymer based on or having acrylamide, methacrylamide, dimethylaminoethyl methacrylate or a derivative of an acrylamide, methacrylamide or dimethyl-aminoethyl methacrylate.

In further embodiments the implant has another outer coating including a degradable polymer, preferably from the class of PLGA (poly(lactic-co-glycolic acid)) or PLGA-PEG block copolymers. In addition, in some embodiments the coating comprises at least one drug.

### DETAILED DESCRIPTION

The object of the present invention is achieved by providing an implant having a base body consisting completely or partially of a biocorrodible metallic material, which has properties such that it decomposes in an aqueous environment to form an alkaline product, and the base body has a coating consisting of or comprising a hydrogel, characterized in that the hydrogel has a reduced swelling capacity at an elevated pH.

One advantage of the inventive approach is that when there is a local increase in pH, the swelling capacity of the hydrogel in the implant coating is reduced. The hydrogel takes up less water at an elevated pH, resulting in a reduction in volume of the hydrogel and thus a decline in volume of the implant coating containing the hydrogel. Subsequently, the hydrogel is then in closer contact around the implant, thus reducing the amount of oxidation partners per unit of time available for corrosion of the implant and thus retarding the corrosion rate of the biocorrodible implant. The local pH can thus normalize again and there are no longer-lasting phases during which the local pH is elevated. The risk of developing an alkalosis is thus definitely reduced. With the use of the inventive implant., the incidence of clinical alkaloses thus also declines. When using the inventive implant, it is thus no longer necessary to counteract a possible alkalosis. e.g., by systemic administration of medicines or drugs. Corresponding drugs may already be embedded in the coating of the biocorrodible implant and are eluted to an increased extent at the site when there is a change in the local pH. Thus, on the whole, definitely smaller doses of drug may be used, which are then preferably made available at the desired site and at the time of need. The patient is less burdened and treatment costs are reduced.

Implants in the sense of the present invention are devices introduced into the body by a surgical procedure and comprise fastening elements for bones. e.g., screws, plates or nails, surgical suture materials, intestinal clamps, vascular clips, prostheses in the area of the hard and soft tissue and anchoring elements for electrodes, in particular pacemakers or defibrillators.

The implant is preferably a stent. Stents of the traditional design have a filigree supporting structure of metallic struts, which are initially in an unexpanded state for introduction into the body and are then widened into an expanded state at the site of application. The stent may be coated before or after being crimped onto a balloon.

According to a first variant, the base body of the implant thus has a coating containing or comprising an inventive hydrogel. A coating in the inventive sense is formed when components of the coating are applied in at least some sections to the base body of the implant. The entire surface of the base body of the implant is preferably covered by the coating. The layer thickness is preferably in the range of 1 nm to 100 μm, especially preferably 300 nm to 30 μm. The amount by weight of inventive hydrogel in the components forming the coating is preferably at least 40%, especially preferably at least 70%. The coating may be applied directly to the implant surface. Processing may then be performed by standard coating methods. Single-layer systems as well as multilayer systems (e.g., so-called base coat layers, drug coat layers or top coat layers) may also be created. The coating may be applied directly to the base body of the implant or other layers may be provided in between, e.g., to improve adhesion.

Alloys and elements in which degradation/rearrangement takes place in a physiological environment are understood to be biocorrodible in the sense of the invention, such that the part of the implant comprising the material is no longer present entirely or at least predominantly. Biocorrodible metallic materials in the sense of the invention comprise metals and alloys selected from the group including iron, tungsten, zinc, molybdenum and magnesium, and in particular those biocorrodible metallic materials which undergo corrosion to yield an alkaline product in an aqueous solution.

The metallic base body preferably consists of magnesium, a biocorrodible magnesium alloy, pure iron, a biocorrodible iron alloy, a biocorrodible tungsten alloy, a biocorrodible zinc alloy or a biocorrodible molybdenum alloy. The biocorrodible metallic material is a magnesium alloy in particular.

A biocorrodible magnesium alloy is understood to be a metallic structure having magnesium as its main component. The main component is the alloy component present in the greatest amount by weight of the alloy. The amount of main component is preferably more than 50 wt %, in particular more than 70 wt %. The biocorrodible magnesium alloy preferably contains yttrium and other rare earth metals because such an alloy is characterized by its physicochemical properties and high biocompatibility, in particular also its degradation products. An especially preferred magnesium alloy has a composition comprising 5.2-9.9 wt % rare earth metals, including 3.7-5.5 wt % yttrium and <1 wt % remainder, where magnesium accounts for the rest of the alloy up to 100 wt %. This magnesium alloy has already confirmed its special suitability experimentally and in preliminary clinical experiments, i.e., it has a high biocompatibility, favorable processing properties, good mechanical characteristics and an adequate corrosion behavior for the intended purpose. In the present case, the collective term “rare earth metals” is understood to include scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The composition of the magnesium alloy is to be selected so that it is biocorrodible. Artificial plasma such as that specified according to EN ISO 10993-15:2000 for biocorrosion investigations (composition: NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) is used as the test medium for testing the corrosion behavior of alloys. A sample of the material to be tested is stored in a defined amount of the test medium at 37° C. in a sealed sample container. At intervals of time—coordinated with the expected corrosion behavior—of a few hours up to several months, samples are taken and tested for traces of corrosion in a known way. The artificial plasma according to EN ISO 10993-15:2000 corresponds to a blood-like medium and offers a possibility for reproducibly simulating a physiological environment in the sense of this invention.

According to the invention, the hydrogel of the implant coating has a reduced swelling capacity at an elevated pH.

An elevated pH in the sense of the invention prevails when the local pH is altered to the basic pH range in comparison with the physiological pH. In particular an elevated pH prevails in the sense of the invention when the local pH is greater than 8.

The term “swelling capacity” in the sense of the invention is understood to be the property of the hydrogel of absorbing a certain amount of water per mmol of hydrogel polymer. A reduced swelling capacity leads to a reduction in the volume of the hydrogel and thus a reduction in the volume of the implant coating containing this hydrogel. Those skilled in the art know of suitable processes and measurement methods for determining the swelling capacity. Measurement methods such as those that have already proven successful in the field of galenics in particular are suitable here.

In a preferred embodiment, the swelling capacity of the hydrogel is reduced at a pH greater than 8.

In especially preferred embodiments, the swelling capacity of the hydrogel is reduced at a pH greater than 8, such that the hydrogel can absorb at least 30% less water per mmol hydrogel polymer than at a physiological pH.

In one embodiment of the invention, the coating of the inventive implant comprises a hydrogel. A hydrogel is a polymer that contains water but is insoluble in water, its molecules being chemically linked, e.g., by covalent or ionic bonds, e.g., by looping the polymer chains to form a three-dimensional network. Inventive hydrogels are capable of changing their volume when there is a change in pH in that they have a reduced swelling capacity at an elevated pH and therefore can absorb less water per mmol of hydrogel polymer. These hydrogels can be produced, for example, by reaction of ethylenically unsaturated monomers and polymers, which have the ionizable groups, with crosslinking agents and polymerization catalysts. As an alternative, suitable hydrogels can be produced by condensation reactions with difunctional and polyfunctional monomers. Those skilled in the art know of suitable monomers and polymers as well as methods of synthesizing same. Likewise, those skilled in the art know of methods and processes for synthesizing suitable hydrogels by means of such monomers and/or polymers.

The hydrogel of the inventive implant coating has pH-dependent swelling properties. The hydrogel preferably has at least one functional group, which is converted from an ionic charge state to a neutral charge state with an increase in pH. At least one functional group of the hydrogel is in an ionic charge state at a physiological pH and is in a neutral charge state at an elevated pH in the sense of the invention. The hydrogel of the inventive implant may have several functional groups that are the same or different, not all of which need be in the same charge state at a physiological pH.

In a preferred embodiment, the at least one functional group is selected from the group comprising an amine group or an amide function.

Preferred hydrogels contain a polymer based on acrylamide, methacrylamide, dimethylaminoethyl methacrylate or a derivative of acrylamide, methacrylamide, or dimethylaminoethyl methacrylate.

In another aspect, the coating of the inventive implant may contain at least one drug. Any known drug may be used as the drug. Such drugs that are suitable for treatment or prevention of an alkalosis are especially preferred. Such drugs are selected from the group comprising vasodilators, anti-inflammatories and drugs for local regulation of pH. Especially preferred drugs are selected from the group containing substances that release NO and bosentan®, dipyridamol, dODN or endothelin receptor antagonists in general, calcium channel blockers such as amlodipine, nifidipine or verapamil.

The inventive implant may have another exterior coating. Such another exterior coating may completely or partially cover the coating comprising the hydrogel. This exterior coating preferably consists of or comprises a degradable polymer, in particular a polymer from the class of PLGA (poly(lactic-co-glycolic acid)) or PLGA-PEG block copolymers. A drug that is freely elutable or is released on degradation of the exterior coating may optionally also be embedded in this additional outer layer. Such an additional exterior coating may be used in a multilayer system to delay the reduction in swelling properties of the hydrogel imparted by the change in pH and to delay the associated volume reduction of the coating layer. The additional exterior coating is first degraded and only then is the inner coating accessible, which then causes a reduction in volume of the hydrogel when an elevated pH prevails.

### EXAMPLES

The invention is explained in greater detail below on the basis of exemplary embodiments.

### Exemplary Embodiment 1—Poly(N-isopropylacrylamide-co-allylamine)

3.8 g (33.6 mmol) N-isopropylacrylamide (NIPAM) and 0.2 g (3.4 mmol) allylamine (10% of the NIPAM monomer) are dissolved in 230 mL at room temperature. Then 0.06% SDS and 0.067 g (1.3 mol %; 0.44 mmol) N,N′-methylenebisacrylamide are added. The solution is degassed with N₂ for 30 minutes while stirring and heated to 70° C. 0.166 g potassium persulfate is dissolved in 20 mL and added to the reaction mixture to initiate the reaction. The reaction is performed for 4 hours at 68-70° C. After cooling to room temperature, the precipitate is dialyzed for five days against water (molecular cutoff (MCO) 13,000 Da). The resulting poly(N-isopropylacrylamide-co-allylamine) has a reduced swelling capacity in an aqueous environment with an increase in pH.

If necessary, a drug may be embedded in the hydrogel.

Matrix preparation and incorporation of drug, if necessary:

1 g of the resulting polymer is mixed with verapamil and crosslinked with 0.04 g (25 wt %) glutaraldehyde for 2 hours at room temperature.

Alternatively, matrix preparation and optional embedding of the drug may be performed as follows: 1 g of the resulting polymer is optionally mixed with verapamil. Then 0.032 g (0.17 mmol) 1-ethyl-3-(3-dimethylaminopropyl)carbodiimide dissolved in 200 μL water and 0.015 g (0.085 mmol) adipic acid dihydrazide, also dissolved in 200 μL water, are added and stirred for 2 hours.

Drugs that react with crosslinking agents such as glutaraldehyde or EDC must be liposomally encapsulated before the reaction.

### Exemplary Embodiment 2—Coating a Stent

A stent of the biocorrodible magnesium alloy WE43 (4 wt % yttrium, 3 wt % rare earth metals. not including yttrium, remainder magnesium and impurities from the production process) is coated as follows:

The stent is cleaned of dirt and residues and clamped in a suitable stent coating apparatus (DES coater, in-house development of Biotronik). With the help of an airbrush system (EFD or Spraying System), the rotating stent is coated on one half side with one of the polymer mixtures from Exemplary Embodiments 1 or 2 under constant ambient conditions (room temperature, 42% atmospheric humidity). At a nozzle spacing of 20 mm, an 18-mm-long stent is coated after approx. 10 minutes. After reaching the intended layer weight, the stent is dried for 5 minutes at room temperature before the uncoated side is coated in the same way after renewed rotation of the stent and renewed clamping. The finished coated stent is dried for 36 hours at 40° C. in a vacuum oven (Vakucell, MMM). The layer thickness of the applied coating is approx. 10 μm.

What is claimed is:

1. An implant with a base body comprising completely or partially a biocorrodible metallic material, where the material has properties such that it decomposes to form an alkaline product in an aqueous environment, and where the base body has a coating comprising or containing a hydrogel, characterized in that the hydrogel has a reduced swelling capacity at an elevated pH.

2. The implant according to claim 1, wherein the implant is a stent.

3. The implant according to claim 1, wherein the biocorrodible metallic material is a magnesium alloy.

4. The implant according to claim 1, wherein the swelling capacity of the hydrogel is reduced at a pH greater than 8.

5. The implant according to claim 1, wherein the swelling capacity of the hydrogel is reduced at a pH greater than 8, such that the hydrogel can absorb at least 30% less water than at a physiological pH.

6. The implant according to claim 1, wherein the hydrogel has at least one functional group which is converted from an ionic charge state to a neutral charge state with an increase in pH.

7. The implant according to claim 6, wherein the functional group is selected from the group comprising an amine function and an amide function.

8. The implant according to claim 1, wherein the hydrogel comprises a polymer selected from the group consisting of acrylamide, methacrylamide, dimethylaminoethyl methacrylate, a derivative of an acrylamide, methacrylamide and dimethyl-aminoethyl methacrylate.

9. The implant according to claim 1, wherein the implant has another outer coating comprising a degradable polymer, PLGA (poly(lactic-co-glycolic acid)) or PLGA-PEG block copolymers.

10. The implant according to claim 1, wherein the coating comprises at least one drug.
